(19) United States
(12) Patent Application Publication
(10) Pub. No.: US 2006/0228734 A1
(43) Pub. Date: Oct. 12, 2006

(54) Fluid processing device with captured reagent beads

(75) Inventors: Charles S. Vann, El Granada, CA (US); Umberto Ulmanella, Foster City, CA (US)

Correspondence Address: KILYK & BOWERSOX, P.L.L.C., 3603 CHAIN BRIDGE ROAD, SUITE E, FAIRFAX, VA, 22030, US

(73) Assignee: Applera Corporation, Foster City, CA (US)

(21) Appl. No.: 11/378,559

(22) Filed: Mar. 17, 2006

Related U.S. Application Data
(60) Provisional application No. 60/663,085, filed on Mar. 18, 2005.

(51) Int. Cl. C12Q 1/68; C12M 1/34

(52) U.S. Cl. 435/6; 435/287.2

(57) ABSTRACT

A fluid processing device, method and system are provided. The fluid processing device can comprise: a substrate; a plurality of reaction regions disposed in or on the substrate; at least one channel interconnecting the plurality of reaction regions, the at least one channel having a cross-sectional area that includes a maximum dimension; and a plurality of reagent-releasing beads. Each reagent-releasing bead can be positioned in a respective one of the reaction regions. Each bead can comprise one or more reaction components for an assay. Each of the reagent-releasing beads can have a minimum dimension that is greater than the maximum dimension of the channel cross-section.

### CROSS REFERENCE TO RELATED APPLICATION

The present application claims benefit under 35 U.S.C. § 119(e) from earlier filed U.S. Provisional Application No. 60/663,085, filed Mar. 18, 2005, which is herein incorporated by reference in its entirety.

### INTRODUCTION

The present teachings relate to a device and method used to load fluids onto a micro-card having a plurality of reaction regions.

### SUMMARY

According to various embodiments, a fluid processing device is provided. The fluid processing device can comprise: a substrate; a plurality of reaction regions disposed in or on the substrate; at least one channel interconnecting the plurality of reaction regions, the at least one channel having a cross-sectional area that includes a maximum dimension; and a plurality of reagent-releasing beads. Each reagent-releasing bead can be positioned in a respective one of the reaction regions. Each bead can comprise one or more reaction components for an assay. Each of the reagent-releasing beads can have a minimum dimension that is greater than the maximum dimension of the channel cross-section.

According to various embodiments, a fluid processing device is provided. The fluid processing device can comprise a substrate, and a pathway disposed in or on the substrate. The pathway can comprise: a loading port, a vent, a first fluid retainment region comprising a reagent-releasing bead in fluid communication with the loading port, a second fluid retainment region comprising a reagent-releasing bead in fluid communication with the vent, and a first channel in fluid communication with the first fluid retainment region and the second fluid retainment region.

According to various embodiments, a method is provided. The method can comprise loading a fluid processing device with a fluid, wherein the fluid processing device comprises a plurality of reaction regions disposed on or in a substrate, interconnected by at least one channel, and each reaction region comprises a reagent-releasing bead comprising a reagent. Each reagent-releasing bead can comprise a reagent-releasing or dissolvable bead and the method can comprise melting or dissolving each reagent-releasing bead. The method can comprise carrying out a reaction process in each of the reaction regions. The method can comprise interrupting fluid communication in the at least channel between at least two of the plurality of reaction regions

It is to be understood that both the foregoing general description and the following detailed description are exemplary and explanatory only and are intended to provide a further explanation of the present teachings, as claimed.

### BRIEF DESCRIPTION OF THE DRAWINGS

The skilled artisan will understand that the drawings, described below, are for illustration purposes only. The drawings are not intended to limit the scope of the present teachings in any way.

FIG. 1 shows a plan view of a fluid processing device, according to various embodiments;

FIG. 2 shows a side plan view of the fluid processing device shown in FIG. 1, according to various embodiments;

FIG. 3 shows a side cross-sectional of the fluid processing device shown in FIG. 1 along line 3-3;

FIG. 4 is an enlarged plan view of a section of the high-density plate of FIG. 5;

FIG. 5 is a plan view of a portion of a high-density plate according to various embodiments;

FIG. 6 is a chart illustrating a diffusion rate of a reporter dye;

FIG. 7 is a chart illustrating a diffusion rate of an amplicon;

FIG. 8 is a perspective view of an embodiment of a fluid processing device manufacturing system illustrating a manufacturing line;

FIG. 9 is a perspective view of an embodiment of fluid processing device illustrating a cover, a plurality of injectors, and a substrate comprising a plurality of channels and reaction regions;

FIG. 10 is a bottom perspective view of the device of FIG. 9;

FIG. 11 is a side cross-sectional view of the fluid processing device of FIG. 9 along line 11-11;

FIGS. 12a-12d are side cross-sectional views of an embodiment of a fluid processing device illustrating a fluid flow through the fluid processing device;

FIG. 13a is a perspective view of an embodiment of fluid processing device illustrating a cover, a plurality of syringes, and a substrate that comprises a plurality of channels and reaction regions;

FIG. 13b is a bottom perspective view of the device of FIG. 13a; and

FIG. 14 is a side cross-sectional view of the fluid processing device of FIG. 13a along line 14-14.

### DESCRIPTION OF VARIOUS EMBODIMENTS

According to various embodiments, a fluid processing device can comprise a micro-card or micro-plate including a plurality of reaction regions. The reaction region can be interconnected by a plurality of channels or flow passageways. A desirably low-cost and high-throughput micro-card type fluid processing device can comprise a plurality of reaction regions, for example, wells. Some or all of the reaction regions can have a volume as small as or even smaller than 1 microliter. Each of the reaction regions can be loaded with different probes, primers, or reagents. Introducing a sample to each of the reaction regions desirably comprises a means for preventing a probe, primer, or reagent in one reaction region from flowing into another fluidly connected reaction region. An undesired fluid flow can contaminate another reaction region, for example, a reaction region downstream of the first reaction region.

According to various embodiments, a fluid processing device is provided. The fluid processing device can comprise: a substrate, a plurality of reaction regions disposed in or on the substrate, at least one channel interconnecting the plurality of reaction regions, and a plurality of reagent-releasing beads. The at least one channel having a cross-sectional area can include a maximum dimension. Each reagent-releasing bead can be positioned in a respective one of the reaction regions. Each bead can comprise one or more reaction components for an assay. Each of the reagent-releasing beads can have a minimum dimension that is greater than the maximum dimension of the channel cross-section.

According to various embodiments, the fluid processing device can comprise a loading port in fluid communication with the at least one channel. The volume of the loading port can be greater than the total volume of all of the plurality of reaction regions and the plurality of channels, combined. The at least one channel can comprise a first end and a second end. The loading port can be in fluid communication with the first end. The fluid processing device can comprise a suction port in fluid communication with the second end of the channel.

In various embodiments, the fluid processing device can comprise a syringe adapted to create suction at the suction port and forming an airtight seal with the suction port. In other embodiments, the at least one channel can comprise a plurality of channels. Each of the plurality of channels can be in fluid communication with a respective plurality of the plurality of reaction regions. Each of the plurality of channels can comprise a first end in fluid communication with the loading port and a second end in fluid communication with the suction port, a vent, and or a capillary vent.

According to various embodiments, each reagent-releasing bead can comprise a material that is substantially non-dissolvable at 25° C. in water and dissolves in water at a temperature greater than about 50° C. Each reagent-releasing bead can comprise a polyethylene glycol. At least one bead can comprise one or more components for real-time fluorescence-based measurements of nucleic acid amplification products held in one of the plurality of reaction regions. One of the plurality of reagent-releasing beads can comprise first and second oligonucleotide primers having sequences effective to hybridize to opposite end regions of complementary strands of a selected polynucleotide analyte segment and a fluorescer-quencher oligonucleotide capable of hybridizing to a analyte segment in a region downstream of one of the primers. The primer can be for amplifying the segment by primer-initiated polymerase chain reaction. The fluorescer-quencher can be for producing a detectable fluorescent signal when an analyte is present in a sample.

According to various embodiments, a substrate can comprise a top surface. The fluid processing device can comprise a cover layer that contacts the top surface and encloses the plurality of reaction regions and the at least one channel. The cover layer can comprise a material that is non-porous, gas-permeable, and liquid-impermeable at pressures of 75 pounds per square inch or less. The cover layer can be optically clear.

According to various embodiments, the substrate can comprise a bottom surface. The fluid processing device can comprise a heat conductive layer having a thermal conductivity of 0.25 Kelvin Watts per meter or greater that contacts the bottom surface. The heat conductive layer can comprise a metal or an alloy thereof. The heat conductive layer can comprise a foil. The heat conductive layer can comprise aluminum, copper, iron, or an alloy thereof. The fluid processing device can comprise a cover layer that contacts the bottom surface and encloses at least one of the plurality of reaction regions or the at least one channel. The bottom cover layer can be a heat conductive layer.

In other embodiments, the loading port can comprise a plurality of loading ports. Each of the plurality of loading ports can be in fluid communication with a respective plurality of the plurality of channels. The plurality of loading ports can be arranged linearly in or on the substrate. A first plurality of the plurality of loading ports can be arranged along a first edge of the substrate and a remaining plurality of the plurality of loading ports can be arranged along a second edge of the substrate. The second edge can be an opposing edge of the substrate.

According to various embodiments, the fluid processing device can comprise a stake disposed in, on, across, or along the at least one channel. The stake can interrupt the interconnecting of at least two of the plurality of reaction regions.

According to various embodiments, the fluid processing device can comprise an excitation beam adapted for optical communication with said components for real-time fluorescence-based measurements of nucleic acid amplification products.

According to various embodiments, the substrate can comprise a micro-plate or card. The at least one channel can comprise a plurality of segments for interconnecting the plurality of reaction regions. Each segment can comprise a serpentine pathway. A stake can be disposed across each segment. A vent can be in fluid communication with one end of the at least one channel and a loading port can be in fluid communication with a distal end of the at least one channel.

In other embodiments, the fluid processing device can comprise a pressure source adapted to interface with the loading port. The loading port can be capable of injecting a first fluid through the at least one channel and the plurality of reaction regions, while replacing a second fluid therein by venting the second fluid from the vent. The first fluid can comprise a liquid and the second fluid can comprise a gas. In other embodiments, the fluid processing device can be disposed in a thermal cycler. In other embodiments, the fluid processing device can be disposed in a fluorescence detection system adapted to perform real-time polymerase chain reaction detection for one of the plurality of reaction wells.

According to various embodiments, a fluid processing device is provided. The fluid processing device can comprise a substrate and a pathway disposed in or on the substrate. The pathway can comprise a loading port, a vent, a first fluid retainment region comprising a reagent-releasing bead in fluid communication with the loading port, a second fluid retainment region comprising a reagent-releasing bead in fluid communication with the vent, and a first channel in fluid communication with the first fluid retainment region and the second fluid retainment region.

According to various embodiments, a method is provided. The method can comprise loading a fluid processing device with a fluid. The fluid processing device can comprise a plurality of reaction regions disposed on or in a substrate, interconnected by at least one channel. Each reaction region can comprise a reagent-releasing bead comprising a reagent. The method can further comprise melting or dissolving each reagent-releasing bead. The method can comprise carrying out a reaction in each of the reaction regions. The method can comprise interrupting fluid communication in the at least channel between at least two of the plurality of reaction regions. In other embodiments, the at least one channel can comprise a plurality of segments interconnecting the plurality of reaction regions. Each segment can have a length long enough to prevent interaction of the reagent in one reaction region of the plurality of reaction regions with the reagent released from another reaction region of the plurality of reaction regions. The method can comprise thermal-cycling the fluid processing device, wherein the reaction process can comprise a polymerase chain reaction. The thermal cycling can comprise raising the temperature of the reagent-releasing beads to a temperature greater than 35° C. and less than 95° C.

In other embodiments of the method, the bead can comprise a water-soluble material, and releasing can comprise heating the bead at a temperature and for a time sufficient to release the reaction components without degrading the reaction components.

According to various embodiments, a different independent reaction can be carried out in each reaction region of a channel, such that the reaction components and/or reaction product of a first reaction region do not contact reaction components and/or a reaction product of an adjacent reaction region. In other embodiments, each of any two adjacent reaction regions can be separated by a channel interval conformation, for example, length and/or depth, sufficient to prevent interaction of released reaction components and/or reaction products from a first reaction region from communicating with released reaction components and/or reaction products from an adjacent reaction region.

According to various embodiments, providing a sample to each of the plurality of reaction regions can comprise providing a sample in a sample port. The sample can be drawn by capillary action through a channel, through some channels, or through all channels to some of the plurality of reaction regions. Each channel can be adapted to draw a liquid sample by capillary action. The channel can be adapted by appropriately configuring the dimensions of the channel. In various embodiments, the channel can comprise a vent at an end.

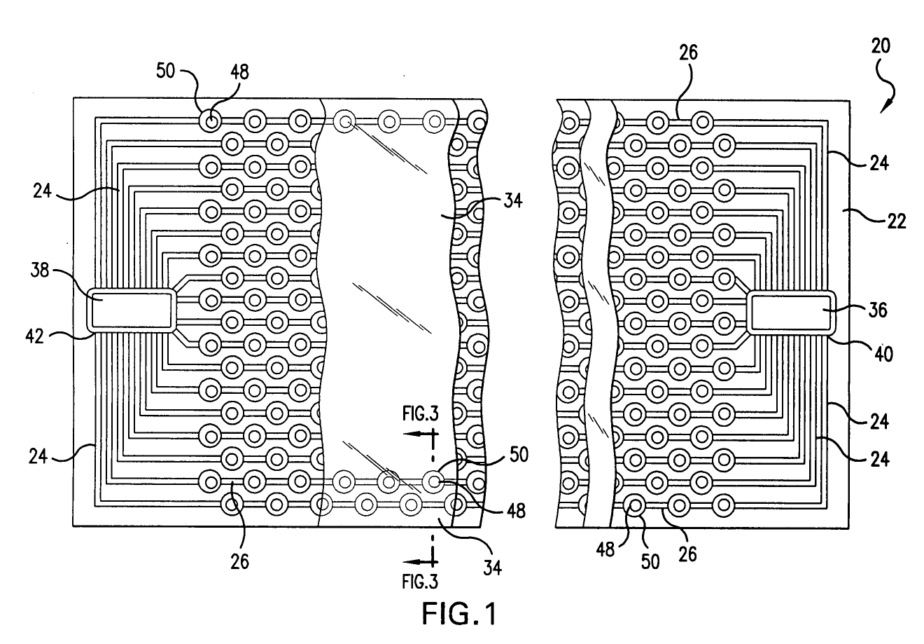

Referring to FIG. 1, a fluid processing device 20 can comprise a substrate 22, a plurality of reaction regions 50, for example wells, formed on or in substrate 22, and a plurality of channels 24, interconnecting reaction regions 50, wherein each of the channels has a cross-sectional area. A plurality of beads 48 can be loaded into the plurality of reaction regions 50, for example, such that one bead is loaded in each region, or more than one bead in each region. Beads 48 can be trapped in place, for example, by a cover 34, for example, an adhesive seal. The beads 48 can be prevented from moving into segments 26 or channels 24. Each bead 48 can comprise a diameter large enough to prevent movement of bead 48 into segments 26 or channels 24. Each bead 48 can comprise biological reagents, for example, probes, one or more unlike primers, chelating agents, enzymes, nucleotides, combinations thereof, or the like.

Fluid processing device 20 can include a sample port 38 formed on or in substrate 22. Sample port 38 can be disposed near a periphery of substrate 22 with sample port 38 being in fluid communication with reaction regions 50 through channels 24. A suction port 36 can be provided on or in substrate 22. Suction port 36 can be disposed near a periphery of substrate 22. Sample port 38 can be disposed on an opposite edge of substrate 22, relative to where sample port 30 is disposed. Suction port 36 can be in fluid communication with reaction regions 50 through channels 24. In the example illustrated, a first end 40 of channel 24 can be in fluid communication with suction port 36, and a second end 42 of the same channel 24 can be in fluid communication with sample port 38.

In various embodiments, bead 48 can comprise a dissolvable material, for example, a material that is solid at ambient temperatures and dissolves at a temperature greater than 25° C. but less than 95° C. The material can comprise polyethylene glycol (PEG). One skilled in the art can modify the chemical structure of PEG, without undue experimentation, such that PEG can be adapted to remain solid or dissolve or melt over a broad temperature range. For example, PEG can be adapted to be solid at an ambient temperature, and therefore the reagents inside bead 48 can remain isolated from one another and from any sample provided through channels 24 to each of reaction regions 50 without cross-contamination from one reaction region to another.

When bead 48 dissolves or melts, reagents contained within bead 48 can be released into a respective reaction region 50. The bead material can comprise a material that will stay melted even during a cooling cycle of a thermal cycler, for example, a cooling cycle having a minimum temperature of approximately 60° C. Bead 48 can comprise a material that does not inhibit a nucleotide amplification reaction or interfere with fluorescent detection that can be carried out to identify components produced during reactions in reaction regions 50.

Sample port 38 can have a volume greater than a total volume of all channels 24 and all reaction regions 50 combined. Suction port 36 can have a volume greater than the total volume of all channels 24 and all reaction regions 50. Channel 24 can comprise a plurality of segments 26. Segments 26 can provide fluid communication between or interconnect reaction regions 50. Segment 26 can be of sufficient length and/or depth to separate different reaction regions 50 such that reagents released from bead 48 in a first reaction region 50 during a thermal cycling process will not come into contact with reagents released from a bead in other reaction regions for a desired number of thermal cycles or duration.

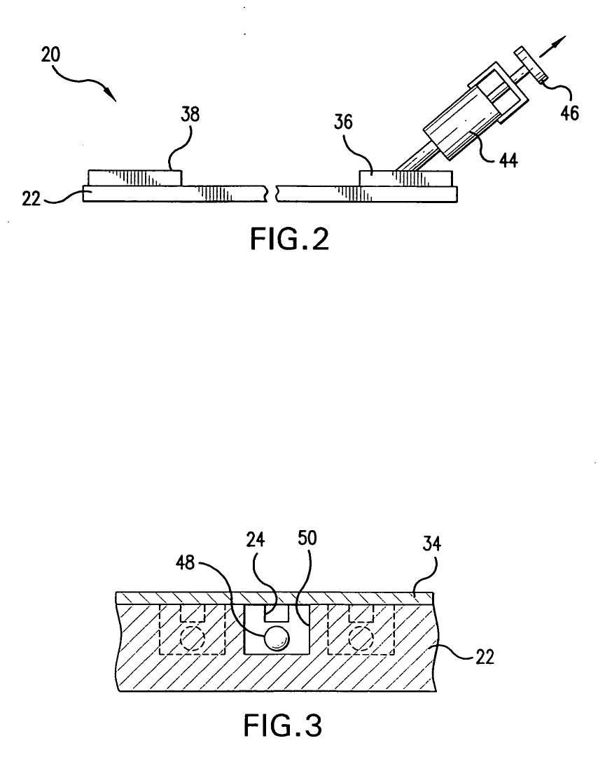

In one embodiment, referring to FIG. 2, a syringe 44 can be provided to form a seal at suction port 36. The seal can be airtight. Syringe 44 can create a pressure differential between suction port 36 and sample port 38. The pressure differential can be formed by extending a plunger 46 of syringe 44. The resulting pressure differential can draw a sample in from sample port 38, through channels 24, through reaction regions 50, to the suction port 36. Alternatively, any other pressure source adapted to create a pressure differential between sample port 38 and suction port 36 can be utilized. When this process of loading a sample from sample port 38 is performed at ambient temperatures, a material adapted to act as a cover or a jacket encapsulating each bead 48 can prevent interaction of the sample with reagents encapsulated in each bead 48.

In other embodiments, channel 24 (FIG. 1) can provide capillary forces sufficient to draw a sample loaded into sample port 38, into channels 24, into reaction regions 50, and into suction port 36.

Utilizing beads 48 to provide reagents can avoid evaporation, dripping, and/or splattering of the components of bead 48 during a loading step. Utilizing beads 48 can provide fixed and/or unknown quantities of reagents to a desired reaction region. The containment or retainment of beads 48 and their associated components at a reaction region during a sample fill or load operation can provide an inexpensive method of filling a sample. Each of the reaction regions can be filled using pressure or capillary forces. The possibility of cross-contamination of reaction regions is eliminated or minimized by providing reagents incorporated or encapsulated in beads 48.

According to various embodiments, a sample can be filled into multiple wells at a time. In other embodiments, a pre-sealed card or fluid processing device can protect pre-filled reagents. Preloaded reagents can be preloaded and/or locked inside a card, for example, Taqman (Applied Biosystems, Foster City, Calif.) reagents. This can prevent a customer or user from using uncertified reagents.

FIG. 3 is a partial cross-sectional view of fluid processing device 20. As shown, channel 24 can have a depth less than reaction region 50. Cover 34 can seal bead 48, channel 24, and reaction region 50.

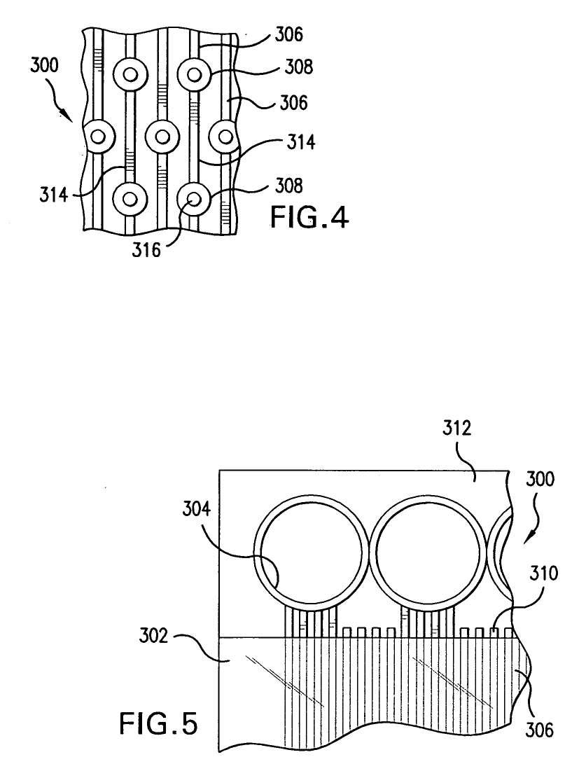

FIGS. 4 and 5 illustrate a partial top-plan view of a fluid processing device 300 according to various embodiments. Fluid processing device 300 can comprise a substrate 312. Channels 306 can be provided on the substrate 312, in substrate 312 (as shown), or both on and in substrate 312. Reaction regions formed along channels 306 are not shown in FIG. 5. A subset of channels 306 can be in fluid communication with a sample port 304. Each channel 306 can comprise a vent 310. Vent 310 can comprise an uncovered area of a distal end of channel 306. In other embodiments, vent 310 can comprise an opening in a cover 302. Cover 302 can be disposed on a surface of substrate 312. Cover 302 can entirely or partially cover channels 306. For example, cover 302 can be provided over channels 306 with the exception that a distal end of channel 306 is not covered, forming vent 310 at the distal end of channel 306. Sample port 304 can remain uncovered. Cover 302 can comprise a film, for example, a polymeric material. Cover 302 can comprise an adhesive backed film. Cover 302 can comprise a non-porous, gas-permeable material.

According to various embodiments, the cover layer can have an exemplary thickness of from about 0.001 inch to about 0.1 inch, for example, from about 0.003 inch to about 0.05 inch. Before, during, or after use, the fluid processing device can be further coated, sealed, or covered by, or can be provided initially coated, sealed, or covered by, a gas-impermeable layer, for example, a non-porous aluminum film layer, a polyolefin film layer, or a polytetrafluoroethylene layer. The gas-impermeable layer can be capable of preventing evaporation, or other loss, or contamination, of a sample within the reaction region.

The non-porous, gas-permeable material of the sealing device, whether in the form of a plug or a cover layer such as a film, sheet, or strip, can include at least one member selected from polysiloxane materials, polydimethylsiloxane materials, polydiethylsiloxane materials, polydipropylsiloxane materials, polydibutylsiloxane materials, polydiphenylsiloxane materials, and other polydialkylsiloxane or polyalkylphenylsiloxane materials. The polysiloxane can be the reaction product of an uncrosslinked reactive polysiloxane monomer and from about 0.01 weight percent to about 50 weight percent polysiloxane crosslinker, for example, from about 0.1 weight percent to about 25 weight percent, or from about 0.5 weight percent to about 10 weight percent polysiloxane crosslinker.

The non-porous, gas-permeable material can include a polysiloxane material, a polyalkylsiloxane material, a polydialkylsiloxane material, a polyalkylalkylsiloxane material, a polyalklyarylsiloxane material, a polyarylsiloxane material, a polydiarylsiloxane material, a polyarylarylsiloxane material, a polycycloalkylsiloxane material, a polydicycloalkylsiloxane material, and combinations thereof. According to various embodiments, the polysiloxane material can include, for example, RTV 615, a polydimethylsiloxane material available from GE Silicones of Waterford, N.Y. The polysiloxane can be formed of a two-part silicone, for example RTV 615.

According to various embodiments, any suitable cover material can be utilized. Exemplary materials can be substantially chemically inert with the reagents placed in the reaction regions. According to some embodiments, a cover material is used that is capable of forming a substantially fluid-tight seal with the upper surface of the substrate, or appropriate regions thereof (e.g., an upstanding rim or lip about the opening of each reaction region). Such a seal can be affected, for example, using conventional adhesives and/or heat sealing techniques. Suitable heat-sealable materials include, for example, polymeric films, such as polystyrene, polyester, polypropylene and/or polyethylene films. Such materials are available commercially, for example, from Polyfiltronics, Inc. (Rockland, Mass.) and Advanced Biotechnologies (Epsom, Surrey England UK).

According to various embodiments, a substantially clear polymeric film can be used, for example, being between about 0.05-0.50 millimeter thick, and that permits optical measurement of reactions taking place in the covered reaction regions. In this regard, it will be recalled that the present teachings contemplate real time fluorescence-based measurements of nucleic acid amplification products (such as PCR). Generally, in such a technique, an excitation beam is directed through the cover into each of a plurality of fluorescent mixtures separately contained in the reaction regions, wherein the beam has appropriate energy to excite the fluorescent components in each mixture. Measurement of the fluorescence intensity indicates, in real time, the progress of each reaction. For purposes of permitting such real time monitoring, each sheet in this embodiment is formed of a heat-sealable material that is transparent, or at least transparent at the excitation and measurement wavelength(s). One suitable heat-sealable sheet, in this regard, is a co-laminate of polypropylene and polyethylene. A heatable platen (not shown) can be used to engage the sheet, once cut and placed over an array of wells, and to apply heat so that the sheet bonds to the substrate.

Other exemplary cover layers that can be used include those described in U.S. patent application Ser. No. 10/762,786, filed Jan. 22, 2004, and in U.S. Patent Application Publication No. US 2003/0021734 A1, published Jan. 30, 2003, which are incorporated herein in their entireties, by reference.

FIG. 4 is an enlarged top-plan view of fluid processing device 300 shown in FIG. 5. Fluid processing device 300 can include channel 306. Channel 306 can be in fluid communication with a plurality of reaction regions 308. Beads 316 can be disposed in or on reaction region 308. Beads 316 can comprise one or more reagent-releasing materials, for example, one or more dissolvable or meltable materials. The beads can comprise, for example, one or more polymers that soften and melt and/or dissolve at elevated temperatures, reverse polymers that soften at lower temperatures, water-soluble materials, and/or solvent-soluble materials, for example, materials that are soluble in acidic solvents, basic solvents, neutral solvents, aqueous solvents, or the like. In some embodiments, the solvent and material remain inert in the presence of a sample and reaction components. Reaction regions 308 can be disposed to maximize the number of reaction sites in or on substrate 312. Reaction regions 308 can be disposed as a grid, for example, a square grid, a rectangular grid, a hexagonal grid, or any other addressable disposition layout known in the art. In some embodiments, reaction regions 308 can be staggered.

Two reaction regions 308 can be separated by a segment 314 of channel 306. Segment 314 can be of sufficient length and/or depth to prevent co-mingling or combination of reagents from a first reaction region 308 into a second adjacent reaction region 308. Each reaction region 308 can comprise a bead or a set of beads 316 disposed therein. Each bead or set of beads 316 can provide biological reagents different from other beads or set of beads 316 disposed in one or more other reaction regions 308 of fluid processing device 300.

According to various embodiments, a sample can be drawn or otherwise forced from sample port 304 into channels 306 and reaction regions 308 by, for example, capillary action or centripetal force. Channel 306 and sample port 304 can be configured to achieve such sample loading. For example, sample port 304 can comprise a depth that can be less than or equal to a depth of the channel 306. For example, channel 306 can comprise a depth of from about 30 μm to about 120 μm, from about 50 μm to about 110 μm, from about 80 μm to about 100 μm, or of about 100 μm. After a sample has been loaded, reaction components or reagents can be released from beads 316. The reagents can interact with the sample disposed in reaction region 308. The reagents can be released from beads 316 by processing beads 316 under conditions effective to melt, dissolve, or otherwise disrupt beads 316 or a layer thereof or thereon, and release reagents contained therein or coated thereon. Such processing can include, for example, heating, thermal cycling, sonicating, cooling, dissolving, or the like. During processing, for example, prior to thermal cycling, sample port 304, channel 306 and reaction region 308 can be covered using a cover as described herein. Prior to processing beads 316, fluid communication through channel 306 can be interrupted, for example, by closing an optical valve, by closing a thermally activatable valve, by closing a deformable valve, by staking, or the like.

According to various embodiments, the fluid processing device can comprise a single-use device. In other embodiments, the fluid processing device can comprise a multi-use device.

According to various embodiments, a method is provided that can comprise providing a fluid processing device comprising at least one channel, a plurality of reaction regions, and two or more beads in communication with a respective reaction region. At least two of the beads can comprise different reaction components and each bead can be of a size sufficient to prevent movement of the bead into the channel. The method can further comprise contacting released reaction components from a bead with a sample to produce a reaction product. The method can comprise, prior to contacting, loading a sample into each of the plurality of reaction regions.

The method can comprise releasing the reaction components from the bead. The bead can comprise a reagent-releasing material, and the releasing can comprise heating the bead to a temperature and for a time sufficient to release the reaction components without degrading the reaction components.

According to various embodiments, a different independent reaction can be carried out in each reaction region of a channel. The reaction components and/or reaction product of a first reaction region can be prevented from contacting reaction components and/or a reaction product of an adjacent reaction region. In other embodiments, the plurality of reaction regions can comprise two adjacent reaction regions separated by a channel of sufficient length and/or depth to prevent interaction of released reaction components and/or reaction products from a first reaction with released reaction components and/or reaction products from an adjacent reaction region.

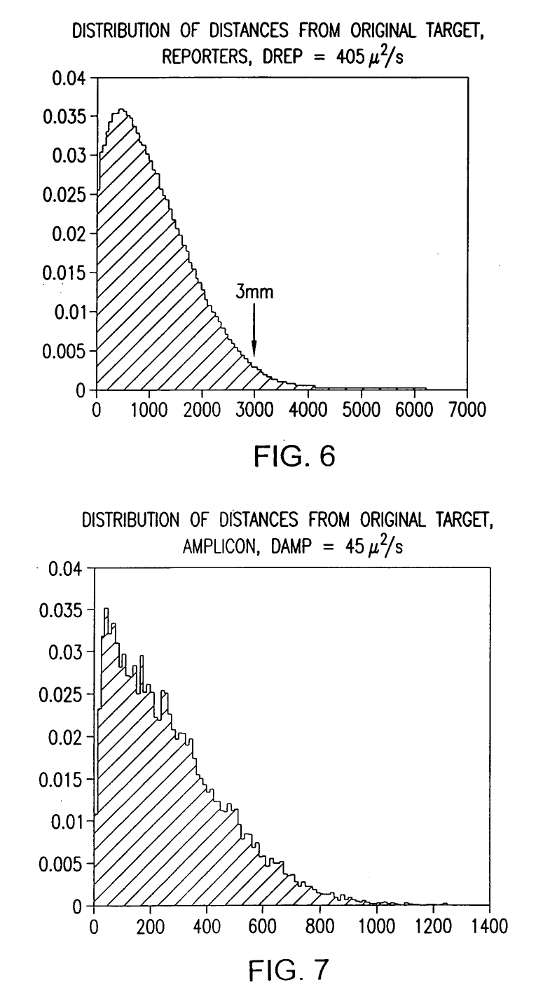

FIGS. 6 and 7 are histograms of the distances that reporters and amplicons respectively are distributed from an origin. The histograms chart a rate of diffusion for an amplicon according to an embodiment of a fluid processing device of the present teachings. The fluid processing device included 6,144 reactions regions. Calculations show that most reporters can diffuse less than two (2) mm in 20 thermal cycles. As can be seen, greater than 80% of the amplicons drift less than 300 micrometers (μm) from an origin. The origin can be a reaction region. A Diffusion Coefficient of an Amplicon (Damp) can be less than 45 μm²/sec. The Damp measures how quickly an amplicon molecule can move per second. Drep is the Diffusion Coefficient for the reporter.

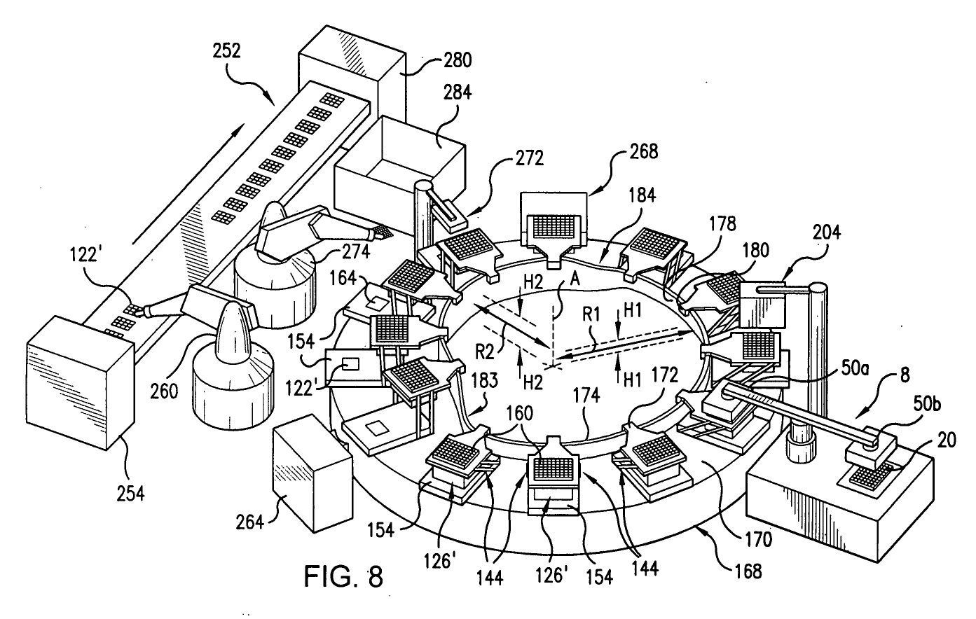

FIG. 8 is a perspective view of a fluid processing device manufacturing system illustrating a bead dispensing system. The bead dispensing system can comprise a bead dispensing system as described in US Patent Application Publication 2003/0021734 A1, published Jan. 30, 2003. A plurality of parallelogram linkage assemblies, such as 144, each carrying a respective conduit assembly 126′, can be seen in combination with a carousel arrangement, denoted generally as 168. Rotational motion of carousel 168 can cause the various linkage assemblies to revolve about the carousel's central axis “A”. Preferably, such motion of the carousel is carried out under the direction of a control computer (not shown). Each conduit assembly is disposed along a region of a respective horizontal link 160 lying radially outward of axis “A”. In one embodiment, for example, each horizontal link is rigidly attached to, or integrally formed with, a frame structure having a central opening (not visible in FIG. 8) configured to receive and support a respective conduit assembly. The other end of each horizontal link 160 rigidly attaches to, or is integrally formed with, a respective elongated arm 172 that extends in the direction toward the rotational axis “A,” reaching to and engaging a rail 174 running along the inner region of the carousel support surface. Rail 174 provides a bearing surface 178, further described below, along which each linkage assembly 144 can ride as it is advanced by carousel 168. In this regard, elongated arm 172 includes a downwardly angled, terminal bend 180 adapted to slide along bearing surface 178. A bearing material can be attached to bend 180 along a region confronting bearing surface 178. Preferably, the bearing material is selected to provide a contact interface with low sliding friction. An exemplary bearing material can be in the form of a boss formed of a low-friction material, such as polytetrafluroethylene (PTFE) or the like, bonded to bend 180 at a region adjacent bearing surface 178.

As mentioned above, and with particular reference to the perspective view of FIG. 8, it can be seen that rail 174 runs along an inner region of the carousel support surface 170. More particularly, the bearing surface 178 of rail 174 includes (i) a first arcuate section disposed a first distance R1 from rotational axis “A” at a first vertical height H1 above the carousel support surface; and (ii) a second arcuate section disposed a second distance R2 from axis “A,” shorter than distance R1, and disposed at a second vertical height H2 that is higher than vertical height H1. The configuration of each such arcuate section is nearly that of a semi-circle, for example, measuring from about 60 degrees to about 85 degrees. Transition sections, as exemplified at 183 and 184, bridge together the first and second arcuate sections. Together, the first and second arcuate sections, and the transition sections, provide a continuous, bearing surface, appearing roughly oblong in top plan view (not shown).

In operation, as each parallelogram linkage assembly 144 is advanced along the first arcuate section of rail 174, a respective conduit assembly 126′ will be located at the lowered position, directly over a substrate 122′. As each parallelogram linkage assembly is moved along the second arcuate section, the respective conduit assembly will be located at the raised position, above and offset from the substrate 122′.

Each reagent-supply location is defined by a well. While only six such locations are shown, arranged side-by-side in a linear fashion, it should be understood that any reasonable number of supply locations can be disposed in any desired spatial configuration. For example, a reagent plate, like plate 20, can include 24, 48, 96, 384, 1024, 1536, or 6144 wells, or more, with each well being configured to support one or more reagent beads. In such arrangements, the wells will typically be arranged in a regular array, e.g., an 8×12, 16×24, 32×32, 32×48, or a 64×96 rectangular array, though other layouts are possible. As indicated above, each reagent-supply location can hold a plurality of beads. Each bead, in turn, can encompass, contain, carry, support, or otherwise include a desired reagent.

Detection instrumentation can be included according to various embodiments, for determining the presence of a bead at target locations of a bead-receiving substrate, such as in the wells of a micro-card. In one embodiment, all beads carrying a particular reagent are formed to display a unique, pre-assigned color. The detection instrumentation, in this embodiment, can be adapted to inspect each target well for a bead of such color. Detection instrumentation can comprise, for example, a CCD camera, a fluorescence detector, a radioactive isotope detector, an RFID detector, an ultraviolet light detector, combinations thereof, and the like.

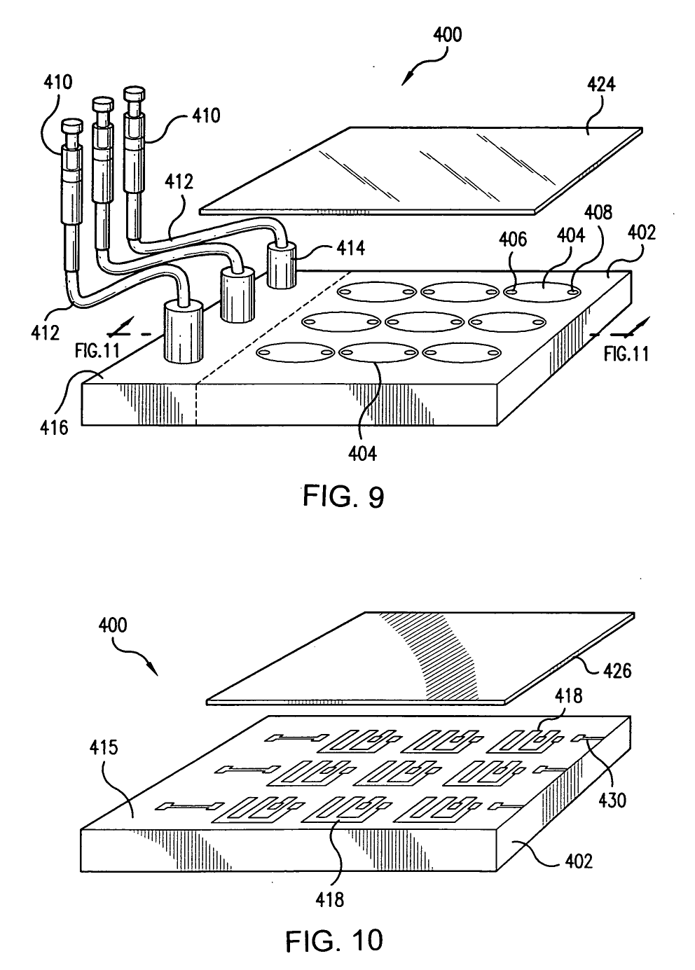

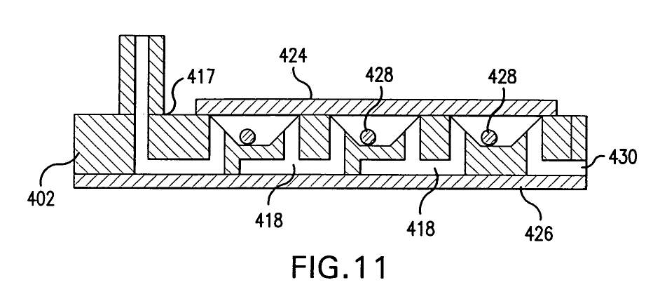

FIGS. 9, 10, and 11 illustrate a fluid processing device 400 according to various embodiments. A syringe 410 can be attached via a tube 412 to a fastener, for example, a Luer lock 414 (as shown) disposed upon a substrate 402. Luer lock 414 can be fluidly connected to an input channel 417. Each input channel 417 can provide a fluid communication between Luer lock 414 and a subset of a plurality of retainment regions 404 defined in or on substrate 402. Each retainment region 404 can comprise an input port 406. Retainment region 404 can comprise an output port 408. Retainment region 404 can be in fluid communication with one or more additional channels. Input port 406 and/or output port 408 can be laser drilled or otherwise formed. Channel 418 can have a serpentine configuration, for example. Channel 418 can provide a fluid communication between two or more different retainment regions 404 via respective input ports 406 and output ports 408. The path of channel 418 can be of sufficient shape and/or dimensions to prevent reagents from a bead in one retainment region 404 from diffusing into another retainment region 404, for example, after melting or dissolving. A vent 430 can be in fluid communication with a subset of retainment regions 404. A continuous fluid flow path that traverses a set of retainment regions 404 and a subset of channels 418 can provide fluid communication from syringe 410 to a respective vent 430 during loading. Subsequent to loading, the vents, channels, reaction regions, or a combination thereof, can be closed or sealed.

Retainment region 404 can retain a bead 428 that can comprise biological reagents. A cover 424 can be placed over a top surface 416 of substrate 402. Cover 424 can seal bead 428 into retainment region 404. Cover 424 can be transparent to allow for optical detection. Cover 424 can be attached to substrate 402 by, for example, adhesion, heat sealing, pressure sealing, combinations thereof, or the like. A seal 426 can be positioned on a bottom surface 415 of substrate 402 as depicted in the bottom view shown in FIG. 10. Seal 426 can be a good heat conductor and can comprise, for example, a metal material, for example, comprising iron, copper, aluminum, and/or comprising thermally conductive carbon particles, and the like. Seal 426 can be scored or creased to form a barrier adapted to interrupt fluid communication through channel 418.

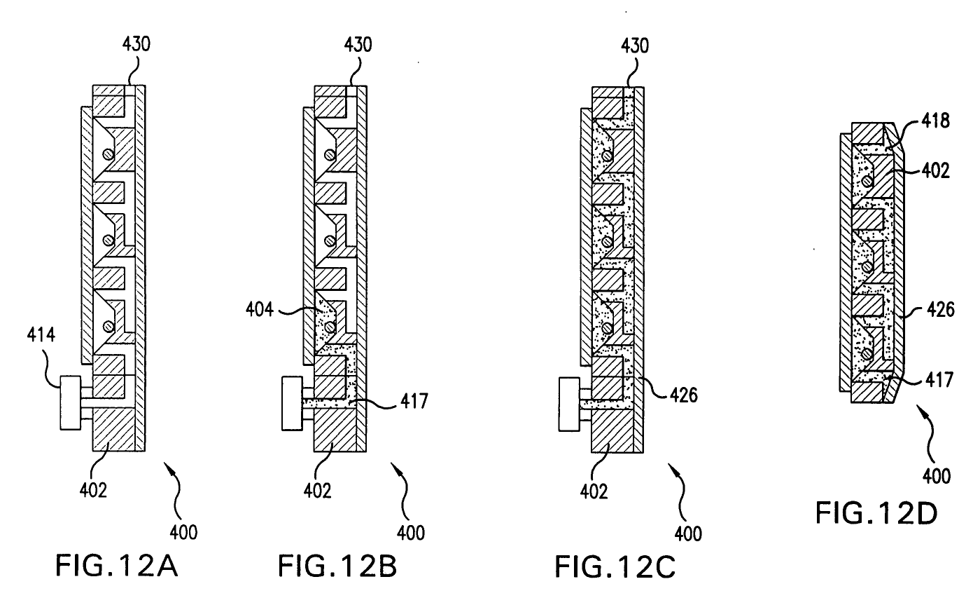

FIGS. 12A, 12B, 12C, and 12D illustrate loading of a sample into a fluid processing device 400. In FIG. 12A, a syringe or pressure source (not shown) can be attached to substrate 402 by twisting the syringe onto Luer lock 414. In FIG. 12B, the syringe forces the sample into input channel 417 and into a first retainment region 404. As the sample is forced into retainment region 404, air can escape through vent 430. FIG. 12C depicts how pressure generated by the syringe has caused the sample to fill a plurality of retainment regions 404, one after another. Pressure exerted by the syringe can be equilibrated or otherwise stopped when the sample exits or reaches vent 430. In FIG. 12D, Luer lock 414 and vent 430 of the substrate have been removed, for example, by cutting off portions of substrate 402, shown at the top and bottom of the figure, comprising vent 430 and Luer lock 414, respectively. The removing can seal-shut input channel 417 and channel 418, for example, by deforming and/or crimping seal 426.

In operation, the fluid processing device can be disposed in thermal contact with a heat source, for example, a thermal cycler. On a first heat cycle, bead 428 can melt, releasing reagents stored in bead 428 into the sample. Over many cycles, reagents released from bead 428 can diffuse from reaction region 404 into channel 418. By design, a length of channel 418 can be sufficient to prevent reagents released from a bead in one retainment region 404 from migrating to an adjacent retainment region 404, for example, over a plurality of cycles, for example, over 20 or more cycles, 30 or more cycles, or 40 or more cycles.

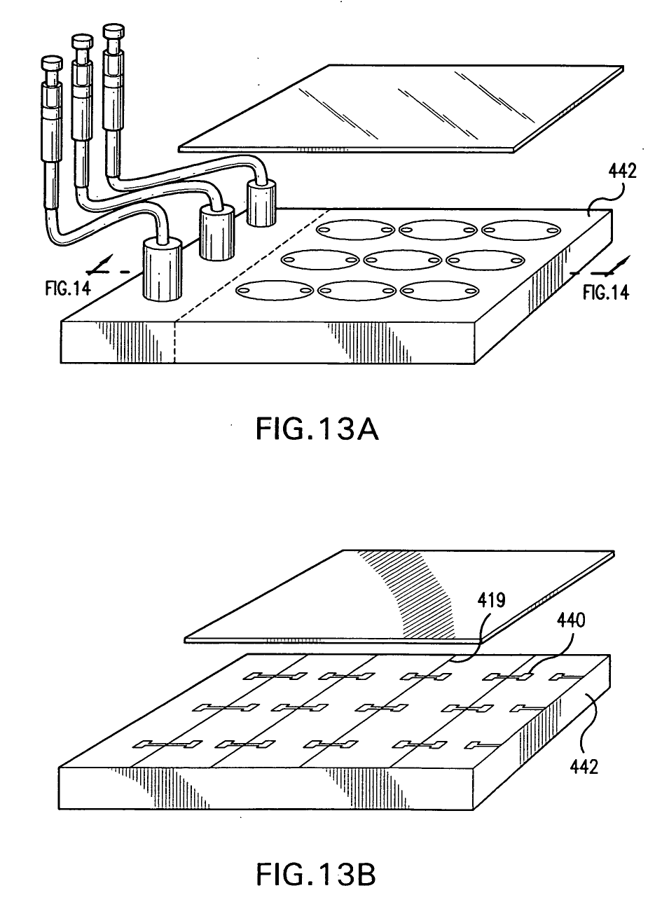

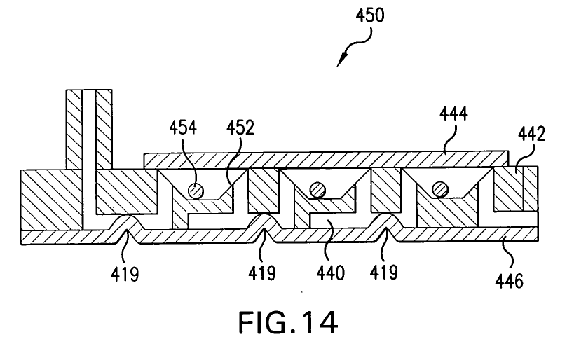

FIGS. 13A, 13B, and 14 show an embodiment of a fluid processing device 450 where channels 440 can be staked, closed, or otherwise interrupted on a bottom surface of a substrate 442 to prevent diffusion of reagents from one retainment region to another. Staking can utilize a physical means of closing a channel, for example, a blade, knife, or other deformer pushed, rolled, or scraped across channel 440. The closing can cause dams 419 to form across the channels 440 in substrate 442. According to various embodiments, the reagents cannot diffuse past dam 419. Reagents can be provided by one or more beads 454 disposed in each reaction region 452, for example, a different bead in each reaction region 452. Reaction regions 452 can be covered with a film 444.

According to various embodiments, closeable valves can be provided between adjacent reaction regions. The closeable valves can comprise an adhesive layer between a cover and a device substrate, such that the adhesive layer can partially define the channel between two reaction regions. The closeable valves can be actuated with a system that comprises a drive mechanism adapted to drive a deformer in a direction towards and into contact with the cover. The deformer can comprise a contact pad or similar compliant device attached at an actuating end thereof.

The drive mechanism can force the contact pad of the deformer into contact with the cover such that the contact pad can mold the adhesive layer into the shape of the underlying channel, to fill-in and close the channel with adhesive. As a result of the compliant or malleable characteristics of the pad, the material of the pad can operate to manipulate the adhesive of the adhesive layer into the channel, thereby closing the valve.

According to various embodiments, the resilient characteristics of the contact pad can allow its shape to change when forced into contact with a structure, such as an adhesive layer valve. The contact pad can be a material that is chemically resistant and inert. The material of the contact pad can be selected to be able to withstand thermal cycling, as can be required while performing PCR. Any suitable elastically deformable and malleable material can be used, for example, a soft rubber, such as silicone rubber. The particular softness characteristics of the contact pad can be chosen depending on the flow characteristics of the adhesive used in the adhesive layer. In other embodiments, the contact pad can have a memory, allowing it to revert back to an original orientation after being forced into contact with the valve. The thickness of the contact pad can be sufficient for the pad to be deformed to an extent such that it can fill an underlying channel. Exemplary of suitable deformable valves that can be used according to various embodiments include those described, for example, in U.S. patent application Ser. No. 10/336,274, filed Jan. 3, 2003, and Ser. No. 10/625,449, filed Jul. 23, 2003, which are herein incorporated by reference.

In some embodiments, the contact pad can be capable of heating the components of an adhesive layer valve. According to various embodiments, the contact pad can heat the adhesive layer, when the contact pad is forced into contact with the valve. For example, the contact pad can be formed partially or entirely of a thermally conductive material or of a material that can act as a resistance heater, or the contact pad can be arranged as a radiant heater, as described in U.S. patent application Ser. No. 10/359,668, filed Feb. 6, 2003, to Shigeura, which is incorporated herein in its entirety by reference. When the contact pad of the deformer is formed of a thermally conductive material, the contact pad can be heated by convection or conduction, for example. When the contact pad of the deformer is made of a material that operates as a resistance heater, it can be heated by running an electrical current through the contact pad, for example. A contact pad formed as a resistance heater can be arranged to include appropriate electrical contacts with a power source.

According to various embodiments, when the contact pad is in position to contact the cover, the temperature of the contact pad can be in a range such that heat transferred to the adhesive layer can reduce the viscosity of the adhesive. By heating and, in turn, reducing the viscosity of the adhesive to promote the manipulability of the adhesive, a heat emitting contact pad can assist in the closing of the valve. Various types of adhesives, for example, pressure sensitive adhesives and hot melt adhesives, can be heated to improve their manipulability.

According to various embodiments, an adhesive layer can be any suitable manipulatable adhesive. For example, pressure sensitive adhesives or hot melt adhesives can be used. Examples of pressure sensitive adhesives include, silicone pressure sensitive adhesives, fluorosilicone pressure sensitive adhesives, and other polymeric pressure sensitive adhesives. Characteristics that can be considered in choosing an adhesive include, for example, tackiness, viscosity, melting point, malleability.

According to various embodiments, the adhesive layer can have any suitable thickness that does not deliteriously affect any sample, desired reaction, or treatment of a sample processed in the device. The adhesive layer can be more adherent to the elastically deformable cover than to the underlying material of the substrate.

According to various embodiments, the fluid processing device can be adapted to match up with a variety of standard format multi-well plates, for example, a 6144 well plate, a 3072 well plate, a 1536 well plate, a 768 well plate, a 384 well plate, or a 96 well plate.

According to various embodiments, a fluid processing device can provide one or more of the following advantages: one-step operation so that a loading port loads multiple wells; liquid volume can be precisely metered to a volume of a well; air bubbles are unlikely; a fluid processing device can be permanently sealed at shipping; a fluid processing device can avoid customer sealing and contamination; a fluid processing device with bead-encapsulated reagents can improve integrity of reagents; a fluid processing device can avoid customer un-sealing and re-sealing of card; a customer can load a sample with a simple, inexpensive syringe; more spacing between wells can enable a better adhesive seal; with more space between wells, fewer bead dispensers can be used; and a fluid processing device comprising beads can be shipped at an ambient temperature.

According to various embodiments, the fluid processing device can comprise at least one heat-actuatable valve arranged in at least one additional flow passageway. The at least one additional flow passageway can be in fluid communication with at least one additional fluid retainment region and at least one of the plurality of fluid retainment regions. The heat-actuatable valve can comprise at least one material selected from a rubber, a plastic, a wax, a paraffin, a polyethylene glycol material, a derivative of a polyethylene glycol material, a polysaccharide, a derivative of polysaccharide, and combinations thereof. The heat-actuatable valve can comprise a material that is insoluble in water at room temperature. The heat-actuatable valve can comprise a material that has a melting point of from about 35° C. to about 95° C., for example, from about 35° C. to about 70° C., from about 35° C. to about 65° C., or from about 35° C. to about 50° C.

According to various embodiments, the fluid processing device comprises one or more beads in each reaction region. Each bead can comprise a substituted polyethylene glycol material, a coated sugar bead comprising reagents in the coating, lyophilized or freeze-dried beads, polysaccharide beads, and the like. An exemplary substituted polyethylene glycol comprises poly (ethylene glycol) methyl ether. In some embodiments, the bead can comprise a polyethylene glycol derivative. An exemplary polyethylene glycol derivative can comprise a triblock copolymer of polyethylene oxide and polypropylene oxide. The bead can comprise a branched polyethylene glycol or derivative thereof. In some embodiments the bead can comprise one or more layers of a reagent-releasing polyethylene glycol derivative coated on top of a different core material. Exemplary substituted polyethylene glycol materials are shown in Table 1 below:

TABLE 1
Examples for Substituted Poly(ethylene glycol)s

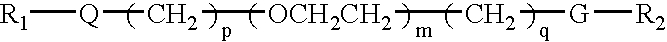

| | Trade | | | | | | | | | mp | ca. | | |
|---|---|---|---|---|---|---|---|---|---|---|---|---|---|
| # | Name | Chemical Name | R₁ | R₂ | G | Q | m | p | q | (° C.) | Mₒ (Da) | HLB | Supplier |
| 2 | Brij ® | poly(ethyleneglycol) cetyl | C₁₆H₃₃ | H | O | O | — | zero | zero | 38-43 | 1124 | 15.7 | ICI |
| | 58 | ether | | | | | | | | | | | Americas, |
| | | | | | | | | | | | | | Norwich, |
| | | | | | | | | | | | | | NY |
| 3 | Brij ® | poly(ethyleneglycol) stearyl | C₁₈H₃₇ | H | O | O | — | zero | zero | 37-39 | 711 | 12.4 | ICI |
| | 76 | ether | | | | | | | | | | | Americas, |
| | | | | | | | | | | | | | Norwich, |
| | | | | | | | | | | | | | NY |
| 4 | Brij ® | poly(ethyleneglycol) stearyl | C₁₈H₃₇ | H | O | O | — | zero | zero | 44-46 | 1152 | 15.3 | ICI |
| | 78 | ether | | | | | | | | | | | Americas, |
| | | | | | | | | | | | | | Norwich, |
| | | | | | | | | | | | | | NY |
| 5 | Brij ® | poly(ethyleneglycol) stearyl | C₁₈H₃₇ | H | O | O | — | zero | zero | 51-54 | 4670 | 18.8 | ICI |
| | 700 | ether | | | | | | | | | | | Americas, |
| | | | | | | | | | | | | | Norwich, |
| | | | | | | | | | | | | | NY |
| 6 | — | Poly(ethylene glycol) | C₁₇H₃₅CO | OCC₁₇H₃₅ | O | O | — | 2 | 2 | 35-37 | 930 | — | Aldrich |
| | | disterate | | | | | | | | | | | Chemical, |
| | | | | | | | | | | | | | Milwaukee, |
| | | | | | | | | | | | | | WI |
| 7 | — | Poly(ethylene glycol) | C₁₇H₃₅CO | OCC₁₇H₃₅ | O | O | — | 2 | 2 | 52-57 | 12500 | — | Polysciences, |
| | | disterate | | | | | | | | | | | Warrington, |
| | | | | | | | | | | | | | PA |
| 8 | — | Poly(ethylene glycol) bis(3- | H₂N(CH₂)₃ | H₂N(CH₂)₃ | O | single | ˜34 | zero | zero | 49 | — | — | Aldrich |
| | | aminopropyl) ether | | | | bond | | | | | | | Chemical, |
| | | | | | | | | | | | | | Milwaukee, |
| | | | | | | | | | | | | | WI |
| 9 | — | Poly(ethylene glycol) | HO₂CCH₂ | CH₂CO₂H | O | single | — | zero | zero | — | 600 | — | Aldrich |
| | | bis(carboxymethyl) ether | | | | bond | | | | | | | Chemical, |
| | | | | | | | | | | | | | Milwaukee, |
| | | | | | | | | | | | | | WI |
| 12 | — | Poly(ethylene glycol) | CH₃ | H | O | O | — | zero | zero | 52 | 2000 | — | Aldrich |
| | | methyl ether | | | | | | | | | | | Chemical, |
| | | | | | | | | | | | | | Milwaukee, |
| | | | | | | | | | | | | | WI |
| 13 | — | Poly(ethylene glycol) | CH₃ | H | O | O | — | zero | zero | 59 | 5000 | — | Aldrich |
| | | methyl ether | | | | | | | | | | | Chemical, |
| | | | | | | | | | | | | | Milwaukee, |
| | | | | | | | | | | | | | WI |
| 14 | — | Poly(ethylene glycol) | CH₃ | CH₃ | O | O | — | zero | zero | 42 | 1000 | — | Aldrich |
| | | methyl ether | | | | | | | | | | | Chemical, |
| | | | | | | | | | | | | | Milwaukee, |
| | | | | | | | | | | | | | WI |

Exemplary derivatives of PEG can include those shown in the Table 2 below:

TABLE 2
Derivatives of PEG*

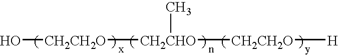

| | Average | | |
|---|---|---|---|
| | Molecular | Melting Pt | |
| Trade name | Weight | (° C.) | HLB |
| Pluronic ® F38 | 4700 | 48 | >24 |
| Pluronic ® F77 | 6600 | 48 | >24 |
| Pluronic ® F87 | 7700 | 49 | >24 |
| Pluronic ® F68 | 8400 | 52 | >24 |
| Pluronic ® F88 | 11400 | 54 | >24 |
| Pluronic ® F127 | 12600 | 56 | 18-23 |
| Pluronic ® F108 | 14600 | 57 | >24 |
| Pluronic ® F98 | 13000 | 58 | >24 |

*Triblock copolymers of PEO and PPO (BASF, Mount Olive, NJ)

The fluid processing device can comprise a plurality of beads, wherein each bead comprises at least one of a polyethylene glycol material, a derivative of a polyethylene glycol material, and a combination thereof. In some embodiments, each of the plurality of beads can include at least one reagent layer or coating that dissolves when contacted with water, for example, after 60 seconds at room temperature, after 30 seconds at 40° C., or after 10 seconds at 50° C. Whether the entire beads, layers of the beads, or other portions of the beads, melt or dissolve, according to various embodiments the degradation of the bead can be attributed to melting, dissolving, or both. In some exemplary embodiments, the beads can each include a reagent layer that can dissolve in water at a temperature of from about 30° C. to about 65° C.

According to various embodiments, a bead can comprise a material having the formula:
R₁-Q-(—CH₂—)ₚ—(—OCH₂CH₂—)ₘ—(—CH₂—)_{q}-G-R₂ Formula 1
wherein:
G and Q are each independently a single bond, O, N, 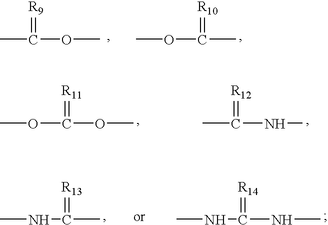
R₁ and R₂ are each independently H, OH, NH₂, CH₃, C₂H₅, OCH₃, OC₂H₅, CH₂OH, —(—CH₂CH₂O—)ₙ—H, CH₂CH₂CH₂NH₂, CH₂CO₂H, C_{g}H_{2g-1}, or CₙH₂ₙ₊₁;
R₉, R₁₀, R₁₁, R₁₂, R₁₃, and R₁₄, are each independently O, S, or NH;
p and q are each independently 0, 1, or 2;
m is an integer from 0 to about 10,000;
at least one of p, q, and m is an integer greater than 0;
g is an integer from 2 to about 20; and
n is an integer from 1 to about 20. The barrier or fluid flow modulator can comprise a material having the formula: 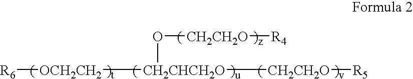
wherein:
R₄, R₅, and R₆ are each independently H, OH, NH₂, CH₃, C₂H₅, OCH₃, OC₂H₅, CH₂OH, —(—CH₂CH₂O—)ₙ—H, CH₂CH₂CH₂NH₂, CH₂CO₂H, C_{g}H_{2g-1}, or CₙH₂ₙ₊₁;
u is an integer from 0 to about 10,000;
g is an integer from 2 to about 20;
n is an integer from 1 to about 20;
t, v, and z are each independently an integer from 0 to about 10,000; and at least one of t, u, and v, is an integer greater than 0. The barrier or fluid flow modulator can comprise a material having the formula:
[R₇—(—CH₂CH₂O—)ₓ—(—CH₂CH₂—)ᵣ—]ₐ-A-R₃—B—[—(—CH₂CH₂—)ₛ—(—CH₂CH₂O—)_{y}—R₈]_{b} Formula 3
wherein:
A and B are each independently a single bond, O, N, 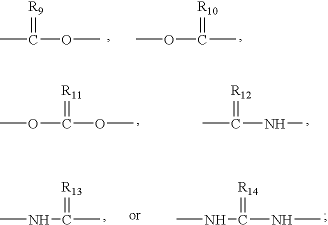
R₇ and R₈ are each independently H, OH, NH₂, CH₃, C₂H₅, OCH₃, OC₂H₅, CH₂OH, —(—CH₂CH₂O—)ₙ—H, CH₂CH₂CH₂NH₂, CH₂CO₂H, C_{g}H_{2g-1}, or CₙH₂ₙ₊₁;
R₃ is CₙH₂ₙ, CₙH₂ₙ₋₂, or CH₂CH(CH₃)O;
R₉, R₁₀, R₁₁, R₁₂, R₁₃, and R₁₄, can each independently be O, S, or NH;
a, b, r, and s are each independently 0, 1, or 2;
x and y are each independently an integer from 1 to about 10,000;
g is an integer from 2 to about 20; and
n is an integer from 1 to about 20. The barrier or fluid flow modulator can comprise a material having the formula: 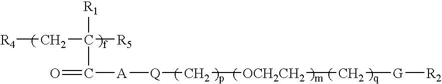
wherein:
A, G, and Q are each independently a single bond, O, N, 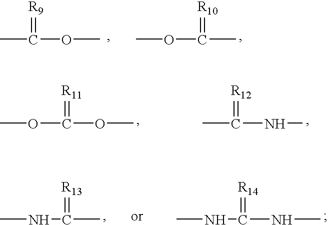
R₁, R₂, R₄, and R₅ are each independently H, OH, NH₂, CH₃, C₂H₅, OCH₃, OC₂H₅, CH₂OH, —(—CH₂CH₂O—)ₙ—H, CH₂CH₂CH₂NH₂, CH₂CO₂H, C_{g}H_{2g-1}, or CₙHₙ₊₁;
R₉, R₁₀, R₁₁, R₁₂, R₁₃, and R₁₄, are each independently O, S, or NH;
f is an integer from 1 to about 10,000;
p and q are each independently 0, 1, or 2;
m is an integer from 0 to about 10,000;
at least one of p, q, and m is an integer greater than 0;
g is an integer from 2 to about 20; and
n is an integer from 1 to about 20.

A wide variety of beads can be used with the present invention. Generally, the beads should resist substantial physical deformations when exposed for a relatively short time to moderately stressful conditions, for example, being pulled upon by an attractive force such as a vacuum, or a magnetic or electrostatic field, as discussed more fully below. Certain embodiments, for example, contemplate the use of beads having a substantially rigid outer shell, or a soft gelatinous coating. Several exemplary types of beads are described next.

In one embodiment, the beads are formed by applying a coating material, such as a gelatin, to a reagent core. The coating cures to form a substantially solid shell about the reagent. The coating can be dissolvable or swellable to permit access to the reagent under controllable conditions (e.g., upon exposure to a particular solvent). Guidance for preparing coated beads, or micro-particles, is provided, for example, in: [1] R. Pommersheim, H. Lowe, V. Hessel, W. Ehrfeld (1998), “Immobilation of living cells and enzymes by encapsulation,” Institut fur Mikrotechnik Mainz GmbH, IBC Global Conferences Limited; [2] F. Lim A. Sun (1980), Science 210, 908; [3] R. Pommersheim, J Schrezenmeir, W. Vogt (1994), “Immobilization of enzymes and living cells by multilayer microcapsules” Macromol Chem. Phys 195, 1557-1567; and [4] W. Ehrfeld, V. Hessel, H. Lehr, “Microreactors for Chemical Synthesis and Biotechtechnology-Current Developments and Future Applications” in: Topics in Current Chemistry 194, A. Manz, H. Becker, Microsystem Technology in Chemistry and Life Science, Springer Verlag, Berlin Heidelberg (1998), 233-252; each expressly incorporated herein by reference.

According to various embodiments, a plurality of bead-like particles act as solid supports for the reagents. For example, reagents can be synthesized on the beads, or absorbed thereto. In still a further embodiment, a slurry or dispersion comprised of a reagent and binding material is used to form a plurality of bead-like particles, with each individual bead having a substantially homogenous consistency. Methods for preparing such beads are well known to those skilled in the art.

A plurality of different reagents can be formed into respective collections or groups of reagent beads, referred to herein as “lots.” For example, 10,000 different reagents can be formed into 10,000 different bead lots, with each lot comprised of a plurality of substantially like beads carrying a respective reagent. To assist in distinguishing beads from different lots, and to provide a means for quickly determining the type of reagent carried by any one particular bead, beads from each lot can be formed to display a particular, pre-assigned color. For example, yellow beads can carry reagent or regent set “A,” blue beads can carry reagent or reagent set “B,” and red beads can carry reagent or reagent set “C.” Beads from each lot can be placed at respective reagent-supply locations.

According to various embodiments, a plurality of bead lots are formed, wherein each bead can comprise, for example, a reagent core covered with a coating material, such as a gelatin or PEG, having well-defined physical and chemical properties. Preferably in this embodiment, all beads in all lots bear substantially the same outer coating (i.e., a “generic” coating), with the coatings for each lot differing only in color, as discussed above. It should be appreciated that this arrangement reduces the risk of equipment contamination due to contact with the reagents themselves. If any residues are left behind as the reagents move through the system, such residues will all be of the same, known coating material. Preferably, the coating material is chosen so that any residues are innocuous to the system. It should further be appreciated that a higher speed for depositing substances can be achieved using such beads, as compared to conventional liquid deposition systems, because the hardware delivering the beads does not require frequent cleaning, nor is time spent aspirating fluids.

While beads of substantially any shape can be used with the present teachings, beads having a generally spherical geometry are particularly well suited for use herein. Also, the system of the invention can be used with beads of various sizes. For example, one embodiment contemplates the use of spherical beads having a diameter of less than about 1 mm. In one such arrangement, each bead can be formed with a diameter of from about 50 to about 500 micrometers, for example, from about 275 to about 325 micrometers. In another embodiment, the beads are larger, such that each bead substantially fills one well of the reagent plate. For example, each bead can have a diameter of between about 1.0-4.0 mm, for example, about 3.7 mm. Each well of the reagent plate, in turn, can be configured with an inner diameter slightly larger than the diameter of a bead. The lower end of each well, in this embodiment, can be shaped to complement the contour of the bead's outer surface.

The beads can carry any desired reagent. As used herein, the term “reagent” can refer to a single substance, or a grouping of substances. According to one preferred embodiment, the reagent carried by each bead includes components useful for real time fluorescence-based measurements of nucleic acid amplification products (such as PCR) as described, for example, in PCT Publication WO 95/30139 and U.S. patent application Ser. No. 08/235,411, each of which is expressly incorporated herein by reference.

According to various embodiments, each bead carries an analyte-specific reagent effective to react with a selected analyte that may be present in a sample. For example, for polynucleotide analytes, the analyte-specific reagent can include first and second oligonucleotide primers having sequences effective to hybridize to opposite end regions of complementary strands of a selected polynucleotide analyte segment, for amplifying the segment by primer-initiated polymerase chain reaction. The analyte-specific detection reagent can further include a fluorescer-quencher oligonucleotide capable of hybridizing to the analyte segment in a region downstream of one of the primers, for producing a detectable fluorescent signal when the analyte is present in the sample.

Rather than relying only upon reflected light to provide a retro-beam from each well, the coating on each bead can be of a type that fluoresces upon being illuminated with light of a certain wavelength. In this way, each bead can generate fluorescent emissions of a particular, pre-assigned color indicative of the reagent that it carries.

Other embodiments of the present teachings will be apparent to those skilled in the art from consideration of the present specification and practice of the present teachings disclosed herein. It is intended that the present specification and examples be considered as exemplary only.

What is claimed is:

1. A fluid processing device, comprising:
a substrate;
a plurality of reaction regions disposed in or on the substrate;
at least one channel interconnecting the plurality of reaction regions, the at least one channel having a cross-sectional area that includes a maximum dimension; and
a plurality of reagent-releasing beads, each reagent-releasing bead being positioned in a respective one of the reaction regions and comprising one or more reaction components for an assay, wherein each of the reagent-releasing beads has a minimum dimension that is greater than the maximum dimension of the channel cross-section.

2. The fluid processing device of claim 1, further comprising a loading port in fluid communication with the at least one channel.

3. The fluid processing device of claim 2, wherein the volume of the loading port is greater than the total volume of all of the plurality of reaction regions and the plurality of channels, combined.

4. The fluid processing device of claim 2, wherein the at least one channel has a first end and a second end, the loading port is in fluid communication with the first end, and the device further comprises a suction port in fluid communication with the second end.

5. The fluid processing device of claim 4, further comprising a syringe adapted to create suction at the suction port and forming an airtight seal with the suction port.

6. The fluid processing device of claim 4, wherein the at least one channel comprises a plurality of channels, each of the plurality of channels is in fluid communication with a respective plurality of the plurality of reaction regions, and each of the plurality of channels has a first end in fluid communication with the loading port and a second end in fluid communication with the suction port.

7. The fluid processing device of claim 1, wherein each reagent-releasing bead comprises a material that is solid at 25° C. and dissolves in water at a temperature greater than about 50° C.

8. The fluid processing device of claim 1, wherein the substrate comprises a top surface and the device further comprises a cover layer that contacts the top surface and encloses the plurality of reaction regions and the at least one channel.

9. The fluid processing device of claim 8, wherein the cover layer comprises a material that is non-porous, gas-permeable, and liquid-impermeable at pressures of 75 pounds per square inch or less.

10. The fluid processing device of claim 1, wherein the substrate comprises a bottom surface and the device further comprises a heat conductive layer having a thermal conductivity of 0.25 Kelvin Watts per meter, that contacts the bottom surface.

11. The fluid processing device of claim 1, wherein the substrate comprises a bottom surface and the device further comprises a cover layer that contacts the bottom surface and encloses at least one of the plurality of reaction regions or the at least one channel.

12. The fluid processing device of claim 2, wherein the at least one channel comprises a plurality of channels, each of the plurality of channels is in fluid communication with a respective plurality of the plurality of reaction regions, and each of the plurality of channels has a first end in fluid communication with the loading port and a second end in fluid communication with a vent.

13. The fluid processing device of claim 1, wherein each of the reagent-releasing beads comprises one or more components for real-time fluorescence-based measurements of nucleic acid amplification products held in at least one of the plurality of reaction regions.

14. The fluid processing device of claim 1, wherein one of the plurality of reagent-releasing beads comprises first and second oligonucleotide primers having sequences effective to hybridize to opposite end regions of complementary strands of a selected polynucleotide analyte segment, for amplifying the segment by primer-initiated polymerase chain reaction, and a fluorescer-quencher oligonucleotide capable of hybridizing to a analyte segment in a region downstream of one of the primers, for producing a detectable fluorescent signal when an analyte is present in an sample.

15. The fluid processing device of claim 1, wherein the at least one channel comprises a plurality of segments for interconnecting the plurality of reaction regions.

16. The fluid processing device of claim 15, further comprising a vent in fluid communication with one end of the at least one channel and a loading port in fluid communication with a distal end of the at least one channel.

17. The fluid processing device of claim 15, further comprising a pressure source adapted to interface with the loading port and capable of injecting a first fluid through the at least one channel and the plurality of reaction regions, while replacing a second fluid therein by venting the second fluid from the vent.

18. The fluid processing device of claim 17, wherein the first fluid comprises a liquid and the second fluid comprises a gas.

19. A system comprising:
a thermal cycler; and
a fluid processing device of claim 1 disposed in the thermal cycler.

20. A system comprising:
a fluid processing device of claim 1 disposed in the thermal cycler; and
a fluorescence detection system adapted to perform real-time polymerase chain reaction detection for one of the plurality of reaction wells.

21. A fluid processing device, comprising:
a substrate; and
a pathway disposed in or on the substrate comprising:
a loading port,
a vent,
a first fluid retainment region comprising a reagent-releasing bead in fluid communication with the loading port,
a second fluid retainment region comprising a reagent-releasing bead in fluid communication with the vent, and
a first channel in fluid communication with the first fluid retainment region and the second fluid retainment region.

22. A method comprising:
loading a fluid processing device with a fluid, wherein the fluid processing device comprises a plurality of reaction regions disposed on or in a substrate, interconnected by at least one channel, and each reaction region comprises a reagent-releasing bead comprising a reagent.

23. The method of claim 22, further comprising releasing reagent from each reagent-releasing bead.

24. The method of claim 22, further comprising carrying out a reaction process in each of the reaction regions.

25. The method of claim 22, wherein the at least one channel comprises a plurality of segments interconnecting the plurality of reaction regions, each segment having a length long enough to prevent interaction of the reagent in one reaction region of the plurality of reaction regions with the reagent released from another reaction region of the plurality of reaction regions.

26. The method of claim 22, further thermal-cycling the fluid processing device.
